(19) United States
(12) Patent Application Publication
(10) Pub. No.: US 2018/0147056 A1
(43) Pub. Date: May 31, 2018

(54) SYSTEM AND METHOD FOR REPLACING AN INFLAMED OR INFECTED HEART VALVE

(71) Applicants: ALEXANDER LAUTEN, KLEINMACHNOW (DE); HANS REINER FIGULLA, JENA (DE)

(72) Inventors: ALEXANDER LAUTEN, KLEINMACHNOW (DE); HANS REINER FIGULLA, JENA (DE)

(21) Appl. No.: 15/879,701

(22) Filed: Jan. 25, 2018

Related U.S. Application Data
(63) Continuation of application No. 15/465,350, filed on Mar. 21, 2017.
Application No. 15/465,350 is a continuation of application No. PCT/EP2015/064745, filed on Jun. 29, 2015.

(30) Foreign Application Priority Data
Sep. 24, 2014 (EP) 14186172.4
Jun. 29, 2015 (EP) PCT/EP2015/064745

(51) Int. Cl. A61F 2/24

(52) U.S. Cl. CPC A61F 2/2418; A61F 2210/0004; A61F 2/2409; A61F 2/2427; A61F 2250/0068

(57) ABSTRACT

The invention relates to a system for replacing a heart valve that is diseased owing to inflammation and/or an infection. The system has: a stent system with at least one expandable stent; and a replacement heart valve which is secured to the at least one stent and has at least two heart valve leaflets. The at least one stent has a coating with an antimicrobial substance or an antimicrobially effective carrier material, preferably on the inner side and/or the outer side.

### CROSS-REFERENCE TO RELATED APPLICATIONS

The present application is a U.S. Continuation Application of co-pending U.S. Continuation application Ser. No. 15/465,350, entitled “SYSTEM AND METHOD FOR REPLACING A HEART VALVE THAT IS DISEASED OWING TO INFLAMMATION OR AN INFECTION,” filed Mar. 21, 2017 (docket number 3050-002-03); which claims priority benefit under 35 U.S.C. § 120 of International Patent Application No. PCT/EP2015/064745 entitled “SYSTEM AND METHOD FOR REPLACING A HEART VALVE THAT IS DISEASED OWING TO INFLAMMATION OR AN INFECTION,” filed Jun. 29, 2015. International Patent Application No. PCT/EP2015/064745 claims priority benefit of European Patent Application No. EP 14186172.4, entitled “SYSTEM FOR REPLACING AN INFLAMED OR INFECTED VALVE OF THE HEART,” filed Sep. 24, 2014. U.S. Continuation application Ser. No. 15/465,350, International Patent Application No. PCT/EP2015/064745, and European Patent Application No. EP 14186172.4 are, to the extent not inconsistent with the disclosure herein, incorporated by reference.

### SUMMARY

The invention relates to a system as well as a method for replacing an inflamed or infected valve of the heart.

An inflammation and/or infection of the heart, so-called endocarditis, is in most cases caused by bacteria such as streptococci, staphylococci or enterococci. Depending on the type of elicitor, life-threatening consequences occur in up to 25% of the cases. Patients who have previously contracted endocarditis also have a greatly increased risk of recurrence. Bacteria enters into the bloodstream for example through injuries in the oral cavity, (post-operative) wounds or in the course of febrile illnesses. In consequence, eddies within the bloodstream, due among other things to a congenital heart defect or calcification, can lead to damaging the endocardium or the heart valves which can provide the genesis for endocarditis.

The prophylaxis as well as the treatment of endocarditis includes the patient taking antibiotics for a sufficiently long period. Due to the low vascularization of heart valve tissue, the body's own immune defense system can only fight bacterial infection of the heart valves to a limited extent. Pathological changes to the heart valves, for example in the form of cicatrization and further degenerations, can develop which can predicate subsequent heart valve insufficiency.

Heart valve insufficiency communicates the insufficient ability of afflicted atrioventricular or semilunar valves to close. During the cyclical diastole of the heart, there is thus an uncontrolled backflow of blood, which causes an insufficient supply of oxygen-rich blood to the body. The body attempts to counter the insufficient blood supply through physiological control mechanisms, which can lead over the medium and long term to further secondary disorders based on hypertrophy of the heart due to reduced cardiac output and high blood pressure amplitudes. Depending on the respectively afflicted heart valve, further symptoms can present such as for example pulmonary edemas or water retention in the limbs.

One possibility for treating endocarditis according to the prior art is surgically removing the inflamed heart valve and implanting an artificial, respectively replacement heart valve. This as a rule entails laborious and costly operations which are coupled with high patient stress and considerable risk. In detail, the patient's chest is opened, the heart stopped by means of a cardioplegic solution, the native heart valve removed and an artificial heart valve sewn to the body's endogenous tissue in its place. Newer methods such as presented in WO2006/076890 A1 provide for transcatheter implantation of artificial heart valves using a stent as a supporting structure.

The present invention is based on the problem as defined of known systems not being suited to treating an endocarditis-afflicted heart as they are not able to deliver antimicrobial, antibiotic, bactericidal and/or comparable active substances to the surrounding tissue and thus do not allow any treating of the inflammatory process. Alongside reduced treatment risk for patients and an optimally precise fitting of a long-lasting replacement heart valve for treating a heart valve insufficiency, there is the related problem of preventing the inflammatory process from causing further damage to cardiac tissue. In addition, approximately 40% of heart valve replacement cases exhibit paravalvular leakage of the implanted system after the transcatheter procedure.

With regard to this problem as defined, the present invention is to fulfill the task of providing an approach for selective interventional treating of an endocarditis-afflicted heart. The present invention must hereby be capable of treating the morbid consequences of endocarditis, in particular cardiac valve insufficiency. The inflammation and/or infection must also be able to be directly treated in situ and the spread of the infection controlled without creating any additional stress or risk for the patient.

This task is thereby solved by the system according to the invention being designed to deliver antimicrobial, antibiotic, bactericidal, anti-inflammatory and/or comparable active substances to the surrounding tissue and into the bloodstream at the site of the affection.

To that end, the system according to the invention comprises a stent/active agent system having at least one stent to which a replacement heart valve is secured. In particular, the claimed system comprises biocompatible materials to ensure that the system will integrate well into the biological environment subsequent implantation. The at least one stent constitutes the carrier and supporting structure of the replacement heart valve and at the same time serves in the positioning and anchoring of the inventive system at the site of implantation. Using a stent-in-stent solution as the stent system is also conceivable. Here, a stent system consisting of more than one stent, in particular 2 or 3 stents, can be provided for use in the inventive system.

The stent/active agent system must be capable of radially displacing the insufficient native heart valve so as to fix the replacement heart valve in its place and guarantee unfailing valve function during cardiac systole and diastole. The at least one stent must also be suited to providing secure retention for the replacement heart valve during the periodic beating of the heart so that the inventive system will not be to able to dislodge from the biological tissue due to the changing pressure conditions in the heart and be flushed from the implantation site. To this end, the inventive system comprises at least one stent which can be expanded by balloon expansion using a balloon catheter and positioned at the site of implantation. The compressed stent lodged within the catheter is expanded by a catheter balloon once filled with a liquid or gas. Alternatively, the at least one stent of the inventive system can be a self-expanding stent. In particular, the stent thereto consists of a shape memory alloy, preferably nitinol. In addition to the shape memory effect at a specific transition temperature close to body temperature, nitinol also exhibits superelasticity, biocompatibility and corrosion resistance. Nitinol is thus already in frequent use in medical technology. The superelasticity is particularly advantageous in terms of the compressed form in which a stent is introduced in the transcatheter method and the expansion at the implantation site. In addition to the two separately implemented expansion methods, it is also possible to combine both methods. Particularly the radial pretensioning force of the stent can be additionally increased post-self expansion by a balloon expansion, whereby higher stability of the inventive system can in turn be achieved in the implanted state.

The replacement heart valve secured to the at least one stent can be a pericardial valve, a porcine heart valve, an artificial heart valve, preferably consisting of biocompatible materials, or a comparable implant or transplant suitable for replacing an insufficient heart valve. The system thus offers the advantage of being able to exhibit the most optimum implementation of a replacement heart valve subject to the patient-specific conditions. The replacement heart valve moreover comprises at least two leaflets. With regard to replacing a three-part heart valve, using more than two, in particular three, leaflets is also conceivable. The use of the inventive system is thus not limited to only replacing an insufficient native aortic valve, particularly not by the number of leaflets.

In their intended use, the leaflets of the replacement heart valve have in particular two positions which they assume during the systole and the diastole of the heart. With the objective of mimicking a native heart valve as its biological model, an equivalent conferring of the leaflet functionality of the biological model is accordingly also conceivable for the further native heart valve replacements. In a first position of the leaflet, during the diastole of the heart, the fluidic connection between the left ventricle and aorta is fully cut off so as to prevent a backflow of blood. The commissures of the leaflets, the internal vascular edges, are thereby in contact with one another. During the systole of the heart, the leaflets assume a second, opened position so that the blood can be pumped from the ventricle into the aorta. The commissures of the leaflets are no longer in contact in this second position.

In one embodiment, the at least one stent has a coating, preferably on the interior and/or exterior side, consisting of an antimicrobial substance or an antimicrobially active carrier material. The at least one stent can thus release antimicrobial agents and achieve improved integrability for the implanted system in contact with the surrounding vascular wall. Particularly the use of an antimicrobial carrier enables a combination with further components seeded at and/or on the carrier material such as anticoagulants or further antimicrobial agents such as bactericidals, etc. Methods of being able to produce such coatings include applying a film to the stent surface as well as further physical and/or chemical deposition procedures for applying a surface coating to the inventive system. It is thus possible to achieve complex release dynamics of preferably antimicrobial active agents on the stent surface.

In a further embodiment, a coating of the stent surface can be activated in controlled manner. Here, a preferably antimicrobial effect does not occur until the surface coating has been activated. Preferably, such a controlled activation of the surface coating can occur upon ultrasound being administered from outside the patient's body, whereby at least one toxic substance, such as for example carbon dioxide, bound to specific carrier media can be released at the stent surface.

Alternatively or additionally hereto, the inventive system comprises a first skirt region within and a second skirt region on the outside of the provided stent, at which a substance filled into the ventricular retention area of the stent is released. Understood in particular by the ventricular retention area in the case of an aortic valve replacement is the retention area of an inventive stent and/or stent system facing the left ventricle of the heart and the aortic retention area positioned opposite thereto. This thus yields a flow of blood through the inventive system, whereby a substance at the system's ventricular blood inlet is released while the bloodstream on the blood outlet-side of the inventive system, at the aortic retention area, passes into the aorta. According to the inventive use of the claimed system, even when replacing other heart valves, the substance release is always to be expected on the blood inlet-side of the inventive system.

In one embodiment of the inventive system, the at least one chamber can be situated in or on the skirt region both at the inner radius (interior side) as well as the outer radius (exterior side) of the at least one stent. Depending on the volume necessary to fill a sufficient quantity of the at least one substance, it is conceivable for, in addition to the volume on the exterior side, a volume for accommodation of a substance to be located on the interior side of the at least one stent. An increase in the substance volume can contribute to maximizing the duration of the substance treatment or to increasing the intensity of the treatment. It is in particular also conceivable for there to be a spatial separation, e.g. by means of an impermeable, permeable or selectively permeable membrane, so that there are in principle two separate chambers at the outer and inner diameter of the at least one stent. Accordingly, a substance not to be released could remain in the chamber at the outer diameter of the at least one stent while a further substance at the inner diameter is released into the bloodstream, particularly for the interventional treatment of endocarditis. There is at the same time also the possibility of implementing diffusion with a permeable or selectively permeable membrane and allowing the substance to only be released by the chamber at the inner diameter of the stent. A chamber extending at the inner and outer radius of the stent thus provides a substantially larger volume for the accommodation and release of substances.

Moreover, a skirt region on the exterior side of the stent with the at least one chamber filled with a substance can concurrently serve as a sealant. In contact with a vascular wall, the system filled with a substance as well as the paravalvular integrity of the system can thus be ensured independent of patient-specific anatomy. In addition to the aortic tissue, the vascular wall also constitutes the biological tissue of a native heart valve as well as the heart in the context of the present invention. In particular, when filled with a substance, the skirt region with the at least one chamber has a volume which is able to prevent an uncontrolled flow of blood at the side edge of the inventive stent during systole and/or diastole. An uncontrolled blood flow hereby refers in particular to the backflow of blood contrary to the anatomically proper direction of flow. Due to a better sealing of the implanted system, its efficiency in terms of imitating a native heart valve concurrently increases. Prevented at the same time is the inventive system dislodging and being flushed from the implantation site, for example due to changing pressure conditions in the heart.

The present invention further provides for the at least one chamber to be able to be filled with a substance prior to and/or during and/or subsequent implantation of the at least one stent. It is thus possible for the at least one chamber to already contain a substance in the compressed state prior to implantation. It is moreover provided for the chamber to be able to be filled with a substance both during as well as subsequent the implantation of the at least one stent. Substances of different types and/or composition can thereby also be filled into the at least one chamber prior to and/or during and/or subsequent implantation. Should there be a plurality of chambers, the present invention also does not preclude the chambers from being filled with different substances or substances of differing compositions. Consequently, it is possible for different active agents to be released, in particular sequentially, and a complex active agent release administered for treating an inflamed or infected heart. A correspondingly versatile and individual patient-specific treatment with active agents can thus be effected in terms of the type and/or intensity and/or duration of the active agent release.

One embodiment of the present invention furthermore comprises a skirt designed as a permeable, in particular selectively permeable, membrane. If a chamber is to be filled with a substance consisting of different fluids and/or components and/or active agents, it is therefore possible for only a portion of the substance filled therein to be released intraoperative and/or postoperative.

Likewise conceivable is the use of multiple fluids of different viscosity or the use of active agents having differing diffusion and/or release characteristics. The therapeutic measures for treatment of an endocarditis-afflicted heart can thus in this way be individually adapted to a patient's own specific conditions. The chamber within the skirt region can also affect the release of a substance in that it comprises, in particular in part, a biologically degradable and/or resorptive material, for example in the form of surface coatings and/or matrices. Accordingly, the release of the at least one substance can in particular commence at a specific time, or after a specific degradation period respectively, or a succession for the sequential release of different substances can be implemented in the inventive system. This thus yields the possibility of a complex treatment strategy for treating endocarditis with the aid of the present invention.

Taking the described release mechanisms into consideration, the present invention exhibits different possibilities for individual release dynamics for a substance filled into at least one chamber. The release dynamics can in particular influence a sequential, intraoperative filling of different components of a substance as well as the specific form and degree of filling of the at least one chamber prior to and/or during and/or subsequent implantation. In addition to the release of substances from the at least one chamber, an at least partial coating of the entire inventive system can at the same time be provided. Integrating resorptive and/or biologically degradable materials as the surface coating and/or matrices, in particular on the at least one stent and/or within the at least one chamber, likewise enables complex release dynamics for active agents. Nor should such components substantially affect the geometrical dimensions of the inventive system in transcatheter implantation.

The present invention additionally provides for an embodiment in which at least one chamber has at least one fluidic connection to the ventricular retention area of the at least one stent. The fluidic connection in particular serves to conduct the substance filled into the at least one chamber to the ventricular retention area of the at least one stent and release it there. It is thereby likewise conceivable for the fluidic connection of a chamber to comprise a longitudinal perforation so that at least some of the substance can exit the fluidic connection toward the ventricular retention area in the course of the flow. Hence, the filled substance can have a direct in situ and preferably antimicrobial effect on the inflammation and/or infections.

In a further embodiment according to the invention, the substance which can be filled into at least one chamber is an antimicrobially active substance and/or comprises at least one antimicrobial component. The substance can thereby comprise fluids and/or components of different viscosities such as for example antibiotics, saline solution, growth factors, anticoagulants, etc. This is particularly to be understood as antimicrobial agents such as e.g. antibiotics for treating endocarditis. Antibiotics thereby generally describe substances of both synthetic as well as biogenic origin, particularly for fighting bacterial infections. Particularly the case of filling the at least one chamber with antimicrobial agents yields the advantage of the inventive system being able to intraoperatively and/or postoperatively treat an inflamed or infected heart with medication following the replacement of an insufficient native heart valve. Depending on the fill substance and its composition, the active substance can thus be released from the inventive system over the short, medium and long term and the patient's morbidity treated. Moreover, it is conceivable for a combination substance of different components and/or fluids to prevent a complete loss of volume of the at least one chamber in that a residual volume remains in the at least one chamber and can thus maintain the paravalvular integrity.

In an additional embodiment, the system according to the invention further comprises a catheter introduction system. Such a catheter introduction system serves in delivering the inventive system to the diseased heart as well as in positioning and anchoring the at least one stent and replacement heart valve affixed thereto at the implantation site by way of balloon expansion and/or self-expansion of the at least one stent. For balloon expansion, a balloon is thereby provided behind the tip of the catheter which is filled with a fluid via a lumen within the interior of the catheter and can be expanded by hydrostatic pressure. Lumens are also essential when using a self-expanding stent and are flushed with cool liquid during delivery of the inventive system to the diseased heart. The self-expansion can be triggered by stopping the flushing of the cool liquid and/or by flushing the lumen with warm liquid. Furthermore, in both expansion procedures, a catheter's provided lumens are preferably continuously flushed so as to prevent blood from entering into the catheter introduction system and/or gas from entering into the patient's vascular system. Depending on the patient's condition and anatomical circumstances, a minimally invasive method for implanting the inventive system can thus be provided, wherein the implantation is accompanied by the lowest possible patient stress, shortened operation time and reduced treatment costs.

The catheter introduction system is moreover suitable for both transfemoral as well as transapical delivery of the at least one stent to the diseased heart. Especially when performing the transfemoral method, the catheter introduction system needs to be flexible, particularly in the distal region of the catheter tip, as well as comprise control elements for controlling and guiding the catheter tip through the patient's vascular system. In addition, the outer diameter of the catheter introduction system is limited to the dimensions of the vascular system in the transfemoral implantation method. The length of the at least one stent and replacement heart valve affixed thereto is likewise limited, as is the length of the catheter tip, so that the inventive system cannot make contact with the endocardium and/or heart muscle of the beating heart in the implanted state.

To fill the at least one chamber of the inventive system with at least one substance, control elements are also provided which are able to selectively fill the at least one chamber with at least one substance. Using external control elements thus yields various advantages for a minimally invasive treatment, particularly less patient stress and lower treatment costs. Should the vascular system or the condition of the patient not be suited to the transfemoral catheter procedure, for example due to the vascular diameters being too small, implantation of the inventive catheter introduction system is also feasible by transapical implantation.

In order to fill the at least one chamber with at least one substance, one embodiment of the catheter introduction system comprises at least one channel for filling the at least one chamber. The channel can be both implemented as a lumen in the catheter introduction system or designed as a separate lumen external of the catheter introduction system. Preferably, however, the channel is provided as a separate lumen external of the catheter introduction system able to be detachably connected to same. Particularly in this preferential embodiment, it is also possible to fill the at least one chamber with a substance subsequent the implantation of the inventive system. To that end, the detachable connection between the catheter introduction system and the at least one channel is disengaged and the catheter introduction system removed from the patient's vascular system. The at least one channel thereby remains within the patient's vascular system and constitutes a continuous fluidic connection for filling a substance into the at least one chamber, preferably by way of the external control elements. It thus also continues to be possible to fill the at least one chamber with a substance subsequent the implantation of the inventive system and the removal of the catheter introduction system. A patient can accordingly also be given preferably antimicrobial agents after the implantation for the purpose of treating an inflammation and/or infection of the heart.

The channel moreover constitutes a fluidic connection which can likewise be designed as tubes or tube connections or other comparable connections of sufficient rigidity and flexibility to supply fluids. The channel can in particular be secured in the chamber by a form-fit and/or frictional connection preferably configured to be disengageable. Accordingly, in one embodiment, the at least one fluidic connection to the at least one chamber can preferably be selectively disconnected by way of a control element of the catheter introduction system.

In the case of an exclusively frictional connection, the fluidic connection can be disconnected by a defined tractive force. Among other things, at least one opening of the chamber can thereby remain through which the at least one substance can exit the chamber and enter into the blood. Alternatively, the at least one opening can be impermeably sealed after the removal of the at least one channel, preferably by a flexible embodiment of the skirt. The advantage results of the catheter introduction system being able to selectively and individually fill a chamber with a substance, whereby the use of the inventive system ensures optimum therapeutic success.

In addition to a system, the present invention additionally claims a method for replacing an inflamed and/or infected heart valve. The inventive system is hereby provided and implanted in order to replace an insufficient native heart valve and treat endocarditis. In particular, implantation of the inventive system provides for delivery to the diseased heart with subsequent expansion and anchoring at the implantation site. Prior to and/or during and/or subsequent the implantation of the inventive system, the at least one chamber can be filled with a substance. It is thereby advantageously possible for a preferably antimicrobial agent to be introduced and released at the implantation site from the at least one chamber of the inventive system on a short, medium and long-term basis.

The following will reference the accompanying drawings in describing the inventive system in greater detail by way of example embodiments. Further embodiments are not to be excluded by the examples specified herein.

### BRIEF DESCRIPTION OF THE DRAWINGS

FIG. 1 is an example embodiment of the catheter introduction system with expanded stent with a replacement heart valve secured thereto;

FIG. 2 is a catheter introduction system with stent and affixed replacement heart valve in accordance with FIG. 1 in the implanted state in the heart;

FIG. 3 is a catheter introduction system with stent and affixed replacement heart valve in accordance with FIG. 2 after the chamber within the skirt region has been filled with at least one substance;

FIG. 4 is a further example embodiment of the inventive system in the form of an expanded stent with a replacement heart valve secured thereto.

### DETAILED DESCRIPTION

In the following detailed description, reference is made to the accompanying drawings, which form a part hereof. In the drawings, similar symbols typically identify similar components, unless context dictates otherwise. Other embodiments may be used and/or other changes may be made without departing from the spirit or scope of the disclosure.

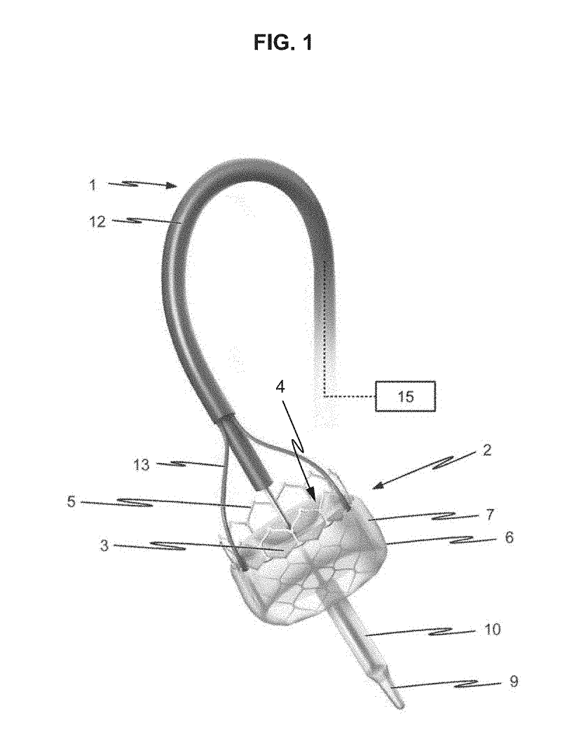

The following will reference FIG. 1 in defining an example embodiment of the inventive system 2 in greater detail. FIG. 1 thereby shows the expanded state of the inventive system 2 comprising a stent 5, a replacement heart valve 3 having at least two leaflets 4 and a skirt region 6 provided in the ventricular retention area of the stent 5 on its interior and exterior side. A chamber 7 which can be filled with at least one substance is shown in the skirt region 6. The depicted catheter introduction system 1 has a catheter 12 with two channels 13 which establish a fluidic connection to the chamber 7 for this purpose.

The catheter introduction system in the depicted embodiment further comprises a balloon 10 for the balloon expansion of the stent 5 and a catheter tip 9. The stent 5 is expanded by a fluid being supplied to the catheter balloon 10 through an inner lumen of the catheter 12 so as to generate enough hydrostatic pressure for the balloon 10 to expand the stent 5. To this end, the catheter can be constructed from a plurality of layers or lumens respectively so as to provide the necessary functionality. Following the expansion of the stent 5, the balloon is compressed again so that the catheter can be removed from the vascular system of the patient at the conclusion of the operation.

The catheter 12 further constitutes a flexible catheter able to be guided through the vascular system of a patient, whereby the tip 9 can be controlled by control elements 15 so as to be able to be guided transfemorally to the heart.

The control elements likewise serve in particular to control the inventive system in conjunction with the catheter introduction system, e.g. for disengaging the catheter 12 from the system 2 after the latter's successful implantation at the implantation site.

To fill the chamber 7 with at least one substance, channels 13 are connected to the chamber 7. The chamber 7 can thereby be in particular sequentially filled with a substance, in particular with a multi-component substance, particularly with different substances. The chamber 7 can furthermore be formed on the exterior and/or interior side of the stent 5. Once the chamber 7 has been filled, the channels 13 can be selectively disengaged from the system 2 by tractive force and/or by means of the control elements 15. The substance filled into the chamber 7 can be released therefrom into the blood of the patient via the remaining open connection points of the channels 13 or by means of diffusion of the substance through the skirt 6.

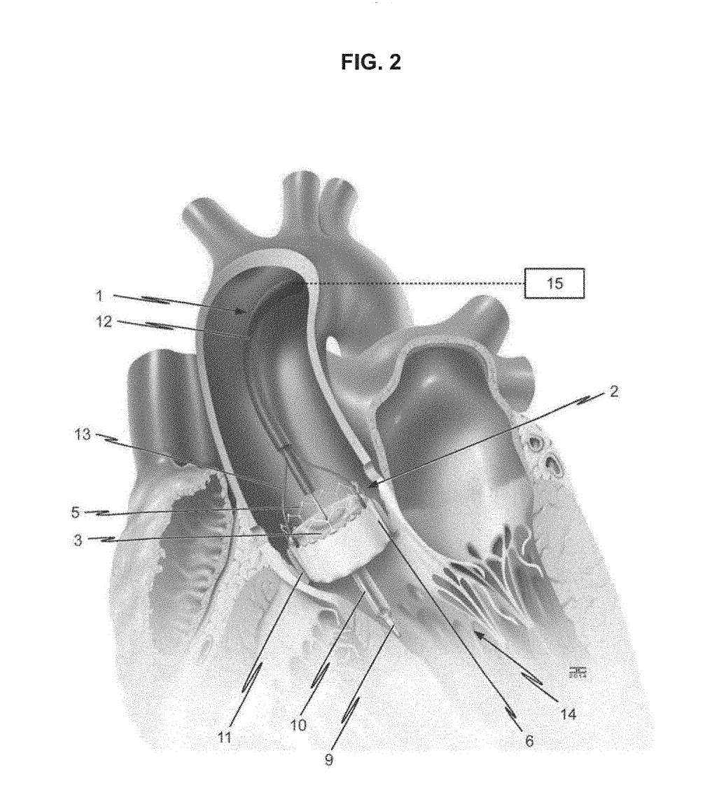

FIG. 2 illustrates the embodiment of the present invention according to FIG. 1 during implantation into a diseased heart 14. In detail, FIG. 2 depicts the expanded stent 5 with the replacement heart valve 2 secured thereto, whereby the insufficient native heart valve 11 is radially displaced by the expanded stent 5. FIG. 2 further illuminates that the length dimension of the system 2 of stent 5, replacement heart valve 2 and catheter introduction system 1 is limited so as to prevent contact with the endocardium of the heart and the heart muscle.

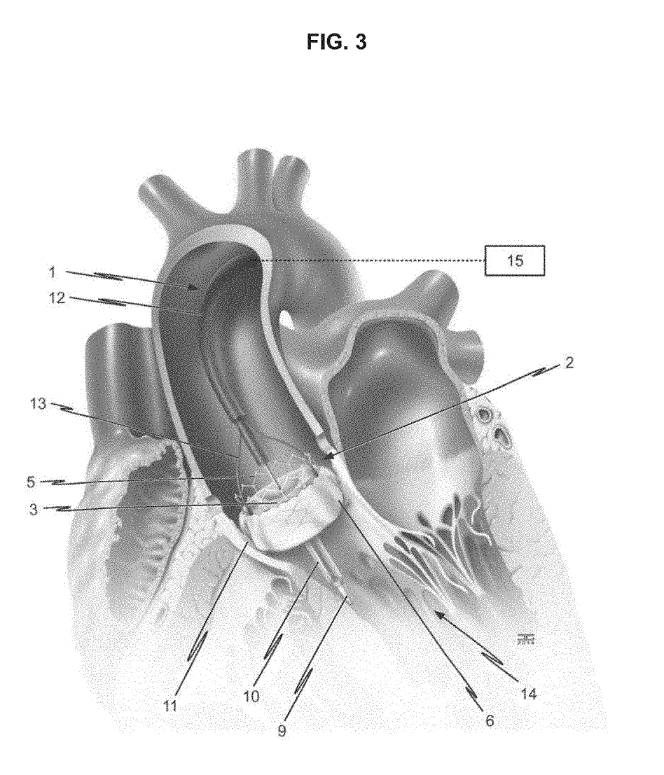

FIG. 3 illustrates the implantation of the system 2 according to FIG. 2, wherein the chamber 7 is filled with at least one substance. It is obvious that by the volumetric expansion of the chamber 7 when filled with at least one substance, the skirt 6 improves the paravalvular integrity in contact with the tissue of the insufficient native heart valve 11. It is in particular likewise possible for the chamber 7 to be filled and only a portion of the introduced substance released so that the substance remaining in the chamber 7 ensures the sealing function of the contact between the skirt 7 and the native heart valve 11. Particularly conceivable is for a substance of multiple components such as e.g. antimicrobial agents, antibiotics, anticoagulants and saline solution to be introduced into the chamber 7, whereby only a portion of the substance is capable of diffusing toward the blood past the permeable or selectively permeable skirt. The skirt 6 exhibits a specific flexibility and elasticity. A further possibility for the selective release of the introduced substance thereby ensues from a pressure-dependent release, wherein the introduced substance 6 is released until the chamber 7 reaches or drops below a hydrostatic pressure limit or the interior of the skirt 6 reaches or drops below a stress limit. The cited example embodiments can ensure the maintaining of a minimum volume in the chamber 7 and the paravalvular sealing function.

FIG. 3 further shows that a substance filled into the chamber 7 can enter directly into the bloodstream and surrounding tissue after being released from the chamber 7 in order to act as an anti-inflammatory there, for example as an antibiotic. The present invention thus provides the advantage of being able to effect interventional treatment of endocarditis directly at its source of inflammation in the diseased heart 14 without subjecting the patient to further stress apart from the transcatheter implantation of a system 2 in accordance with the invention. In addition to the release of medications such as, for example, various antibiotics, use to release anticoagulants or other substances for interventional therapy is also equally possible. In addition to the at least one chamber 7 filled with substances, the present invention furthermore also allows for surface coatings of the system 2 and biologically degradable material, e.g. in the form of resorptive matrices, within the chamber 7 as further release mechanisms for interventional therapy.

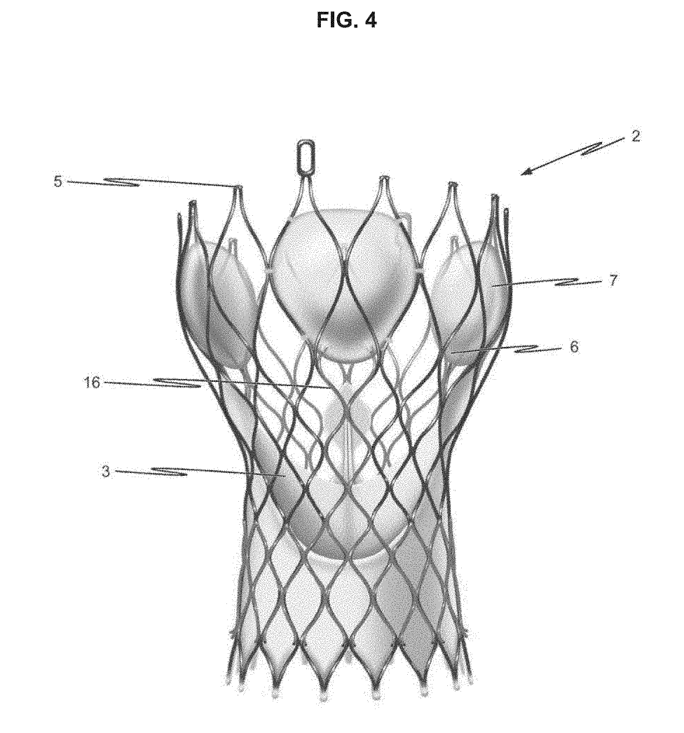

FIG. 4 illustrates a further embodiment of the inventive system 2 in the expanded state of the at least one stent. Here, a replacement heart valve 3 is secured to a stent 5. A skirt region 6 is moreover provided in the aortic retention area of the stent 5, wherein a plurality of individual chambers 7 are in this case provided on the interior side of the stent 5. Each of the chambers 7 thereby has at least one fluidic connection 16 to the ventricular retention area of the stent 5 in order to enable the release of a substance filled into the chambers at that point. The respectively introduced substance flows out of the chambers via the fluidic connections 16 and preferably disperses into the branches of the fluidic connections 16. A distributed release of the substance over the entire extent of the stent 5 is thus enabled. The chambers 7 as well as the fluidic connections 16 are thereby fixed to the stent 5 by means of clamping, sewing or other comparable attachment option.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A method for replacing or repairing an inflamed or infected heart valve,
wherein the inflammation or infection is directly treated in situ and a spread of the inflammation or infection is controlled in situ by means of an active agent implant.

2. The method according to claim 1,
wherein antimicrobial, antibiotic, bactericidal, anti-inflammatory and/or comparable active substances are delivered by means of the active agent implant in or at the native infected heart valves to release these substances into the surrounding tissue and into the bloodstream at the site of affection.

3. The method according to claim 2,
wherein the active agent implant comprises:
at least one transcatheter implantable heart valve; or
at least one artificial replacement heart valve; or
at least one surgically replaceable heart valve; and
wherein the active agent implant has a coating of an antimicrobial substance or an antimicrobially active carrier material.

4. The method according to claim 3, wherein the coating of the antimicrobial substance or antimicrobially active carrier material is disposed on the interior side of the active agent implant.

5. The method according to claim 3, wherein the coating of the antimicrobial substance or antimicrobially active carrier material is disposed on the exterior side of the active agent implant.

6. The method according to claim 3,
wherein the antimicrobial coating of the active agent implant is activated in a controlled manner subsequent the implantation.

7. The method according to claim 3,
wherein the active agent implant comprise a skirt region, wherein at least one first area of the skirt region is provided on the interior and/or at least one second area of the skirt region is provided on the exterior of the active agent implant, wherein at least one chamber fillable with a substance is provided in or on the skirt region and designed such that the substance accommodated in the chamber of the at least one implant is selected to be released to the surrounding tissue.

8. The method according to claim 7,
wherein the at least one chamber is configured in the skirt region in the first area on the interior side and/or in the second area on the exterior side of the active agent implant.

9. The method according to claim 8,
wherein the chamber is filled with a substance prior to and/or during and/or subsequent the implantation of the at least one implant.

10. The method according to claim 7,
wherein the skirt region comprises a permeable membrane.

11. The method according to claim 10,
wherein the permeable membrane comprises a selectively permeable membrane.

12. The method according to claim 7,
wherein the skirt region comprises an impermeable membrane.

13. The method according to claim 7,
wherein the at least one chamber has at least one fluidic connection to a ventricular retention area of the active agent implant.

14. The method according to claim 2,
wherein the antimicrobial substance or antimicrobial carrier material comprises an antimicrobially active component.

15. A method for replacing an inflamed or infected valve of the heart,
wherein the method comprises:
implanting an active agent implant having at least one heart valve into the diseased heart for the replacement of a diseased native or artificial heart valve; and
wherein at least one chamber of the active agent implant is filled with a substance prior to, during, and/or subsequent the implantation of the active agent implant.

16. A method for replacing an inflamed or infected valve of the heart, comprising:
providing a system including:
a stent system further including at least one expandable stent;
a replacement heart valve secured to the at least one expandable stent and comprising at least two heart valve leaflets;
a coating of an antimicrobial substance or an antimicrobially active carrier material on an interior side and/or an exterior side of the at least one expandable stent; and
a chamber fillable with a releasable substance, provided in or on a skirt region of the stent system;
filling the chamber with the releasable substance such that the releasable substance is releasable from the chamber to surrounding tissue; and
implanting the stent system including at least one expandable stent into a diseased heart for the replacement of the inflamed or infected valve of the heart.

17. The method according to claim 16,
wherein the chemical substance comprises a polymer.

18. The method according to claim 16,
wherein the antimicrobially or anti-inflammatory active agent comprises at least one of antibiotics and microparticles.

19. The method according to claim 16,
wherein the timeframe of controlled local release of the antimicrobially or anti-inflammatory active agent is between immediate and several months.

20. The method according to claim 19,
wherein the timeframe of controlled local release of the antimicrobially or anti-inflammatory active agent varies from immediate to several months depending on the underlying disease process.
